(19) United States
(12) Patent Application Publication
(10) Pub. No.: US 2016/0136186 A1
(43) Pub. Date: May 19, 2016

(54) DIRECT MEDICAMENT PERIODONTAL DELIVERY METHOD AND ASSEMBLY FOR REDUCTION OF THE SYSTEMIC INFLAMMATORY MARKERS

(71) Applicant: Duane C. Keller, St. Louis, MO (US)

(72) Inventor: Duane C. Keller, St. Louis, MO (US)

(21) Appl. No.: 14/946,622

(22) Filed: Nov. 19, 2015

Related U.S. Application Data
(60) Provisional application No. 62/081,943, filed on Nov. 19, 2014.

(51) Int. Cl. A61K 31/65; A61K 9/00; A61K 33/40

(52) U.S. Cl. CPC A61K 31/65; A61K 33/40; A61K 9/0063

(57) ABSTRACT

Methods and systems for reducing an identified systemic inflammatory marker associated with cardiovascular disease in a patient is provided through periodontal treatment that includes identifying an oral periopathogen related to the systemic inflammatory marker and a biofilm and an environment associated with the periopathogen, preparing a periodontal tray for direct application of a selected medicament having a efficacy in regard to the identified oral periopathogen and biofilm of the gum tissue associated with the one application region in which the medicament is to be placed and applying the tray with the placed selected medicament to the treatment region of the patient such that at least a portion of the selected medicament is forced into a gingival sulcus or periodontal pocket of the gum tissue of the treatment region and monitoring and continuing treatment to reduce the identified systemic inflammatory marker.

### CROSS-REFERENCE TO RELATED APPLICATIONS

This application claims the benefit of U.S. Provisional Application No. 62/081,943, filed on Nov. 19, 2014, the disclosure of which is incorporated herein by reference.

### FIELD

The present disclosure relates to methods for treating periodontal disease and, more specifically, to assemblies and methods of reducing the systemic inflammatory marker through periodontal disease treatments.

### BACKGROUND

The statements in this section merely provide background information related to the present disclosure and may not constitute prior art.

Periodontal disease has been associated as a co-contributor for inflammatory diseases including cardiovascular disease, atherosclerosis stroke, type 2 diabetes, arthritis and other systemic inflammations. Inflammatory markers such as lipoprotein-associated phospholipase A2 (Lp-PLA2), Interleukin 1 (IL-1), Interleukin 6 (IL-6), Interleukin 8 (IL-8), and others along with tumor necrosis factor-alpha (TNF-alpha), C-reactive protein, plasminogen activator inhibitor type-1 and other markers demonstrate an increased risk of heart attack and ischemic stroke. It is not known to what extent treating periodontal disease may affect changes in these markers.

Measuring systemic inflammatory markers in blood levels evaluates specific inflammatory enzymes and substances that participates in systemic inflammation and has been associated with a significant increased risk for systemic inflammation including heart attack and stroke that has been found to be associated with periodontal inflammation, indicating that periodontal treatments could reduce the risk of systemic inflammatory disease, including cardiovascular diseases. The recognition of this association has led investigators to determine systemic inflammatory marker levels decrease following periodontal surgical and phase I periodontal therapy.

However, very little has been done to develop periodontal disease treatment methods and assemblies that proactively treat periodontal disease as a treatment for lowering the risk of systemic inflammatory risks in patients.

### SUMMARY

The inventor hereof has succeeded at designing methods and assemblies for reducing the risk of systemic inflammation risks such as those associated with cardiovascular disease through treatment of periodontal disease focused on lowering of systemic inflammatory markers including, but not limited to, lipoprotein-associated phospholipase A2 (Lp-PLA2) blood levels and other systemic markers such as, Interleukin 1 (IL-1), Interleukin 6 (IL-6), Interleukin 8 (IL-8), tumor necrosis factor-alpha (TNF-alpha), C-reactive protein, and plasminogen activator inhibitor type-1 (referred herein individually and collectively as “systemic inflammatory markers”), through treating and controlling oral periopathogens in patients. Such periodontal treatment aimed at lowering of systemic inflammatory markers and substances provides enhancements and unexpected benefits to patients beyond preventing the periodontal disease and loss of gums and teeth, to include a reduction in the risk of cardiovascular disease, which heretofore, was never an objective or focus of periodontal treatments.

In one aspect, a method of reducing a systemic inflammatory marker associated with cardiovascular disease in a patient is provided through treatment of periodontal disease that includes performing a medical evaluation examination of the patient that includes, among other processes, the determining of a level of one or more systemic inflammatory markers for cardiovascular disease in the patient. The method includes comparing the determined level of the systemic inflammatory marker to a predetermined level of the systemic inflammatory marker and where the comparing results in a determined systemic inflammatory marker level being greater than the predetermined level, the process includes performing a periodontal evaluation examination of a patient. Such evaluation includes evaluating a gum tissue associated with one of more teeth of the patient and identifying an oral periopathogen and a biofilm and an environment associated with the periopathogen, as well as identifying one or more treatment regions related to the identified oral periopathogen and the biofilm. The method further includes preparing a periodontal medicament delivery tray for the patient with the delivery tray having at least one application region including at least one tooth indentation for direct application of one or more medicaments to at least one of the identified application regions. Next, the method includes selecting a medicament for placement in one of the application regions of the periodontal medicament delivery tray where selecting is a function of a efficacy (known, predetermined or discovered) of the medicament in regard to the identified oral periopathogen and biofilm of the gum tissue associated with the one application region in which the medicament is to be placed. The selected medicament can include one or more agents, and includes at least one oxygenation agent among such one or more agents. The method also includes placing the selected medicament into the associated application region of the prepared tray, applying the tray with the placed selected medicament to the treatment region of the patient for a predetermined period of time such that the medicament is directly delivered or applied against the gum tissue of the treatment region and at least a portion of the selected medicament is forced into a gingival sulcus or periodontal pocket of the gum tissue of the treatment region. As with most treatments, a single treatment is not enough and as such, the method includes repeating the performing of a medical evaluation examination, the comparing selecting, placing and applying process steps until at least one occurs, a) the comparing results in the determined systemic inflammatory marker level being less than or equal to the predetermined level, b) during the periodontal evaluation examination the evaluating of the gum tissue identifies the oral periopathogen is less than a predetermined level, c) during the periodontal evaluation, it is determined that the identified environment for the identified periopathogen has been substantially altered from the first performed medical evaluation examination.

In another aspect, which is one embodiment that is similar to the above, a method of periodontal treatment provides for lowering an exemplary one of the systemic inflammatory markers, in this case lipoprotein-associated phospholipase A2 (Lp-PLA2) level in blood of a patient. Such an exemplary embodiment includes performing a medical evaluation examination of a patient that includes determining a level in the blood of the patient of lipoprotein-associated phospholipase A2 (Lp-PLA2) level and comparing the determined level of Lp-PLA2 to a predetermined level of Lp-PLA2. Where the comparing process or step results in the determined level of Lp-PLA2 is greater than the predetermined level of Lp-PLA2, the method includes performing a periodontal evaluation examination of a patient that includes evaluating a gum tissue associated with one or more teeth of the patient and identifying an oral periopathogen and a biofilm and an environment associated with the identified oral periopathogen, as well as identifying one or more treatment regions related to the periopathogen, biofilm and environment. A periodontal medicament delivery tray is prepared for the patient to have at least one application region including at least one tooth indentation for direct application of a medicament to at least one of the identified application regions. The method includes selecting a medicament for placement in at least one of the application regions of the periodontal medicament delivery tray where the selected medicament includes at least an oxygenation agent and an antibiotic and such selecting is a function of a predetermined efficacy of the medicament in regard to the identified oral periopathogen and biofilm. The selected medicament is placed in the associated application region of the prepared tray and the prepared tray with the placed selected medicament is applied to the treatment region of the patient for a predetermined period of time. This applying includes directly delivering the placed selected medicament against the gums of the treatment region and forcing at least a portion of the selected medicament into the gingival sulcus area or periodontal pocket of the gum tissue of the treatment regions. The steps of performing a medical evaluation examination, the comparing, the selecting, the placing and the applying process steps may be repeated one or more times until at least one occurs, a) the comparing results in the determined level of Lp-PLA2 being less than or equal to the predetermined level; b) during the periodontal evaluation examination the evaluating of the gum tissue identifies the oral periopathogen is less than a predetermined level, c) during the periodontal evaluation, it is determined that identified environment for the identified periopathogen has been substantially altered.

This disclosure discusses treating patients periodontal conditions with the Perio Protect Method (PPM) and evaluating systemic inflammatory markers, before, during and after periodontal treatment. This disclosure is based on pilot experimentation study demonstrating treatment of patient with periodontal conditions and initial results that indicate that the described treatment results in decreasing the periodontal markers and also the lowering of systemic inflammatory markers that are believed to be important as an adjunct of cardiovascular treatment.

Various aspects of the present disclosure will be in part apparent and in part pointed out below. It should be understood that various aspects of the disclosure may be implemented individually or in combination with one another. It should also be understood that the detailed description and drawings, while indicating certain exemplary embodiments, are intended for purposes of illustration only and should not be construed as limiting the scope of the disclosure.

### BRIEF DESCRIPTION OF THE DRAWINGS

FIG. 1 is a cross-sectional view of a tooth and gingival tissue that may have a periodontal disease.

FIG. 2 includes FIG. 2A that is a top perspective view of a periodontal medicament delivery tray, FIG. 2B is top plan view of the periodontal medicament delivery tray and FIG. 2C is a side elevational view of a periodontal medicament delivery tray as worn by a patient for use in one or more embodiments of the treatments of the present disclosure.

FIG. 3 includes FIG. 3A that is a cross-sectional view of a positive pressure medicament delivery tray placed about a tooth and associated gum tissue according to one embodiment, FIG. 3B is a cross-sectional view of a second embodiment of a positive pressure tray placed about a tooth and associated gum tissue, and FIG. 3C is a third embodiment of a medicament tray placed about a tooth and associated gum tissue.

FIG. 4 is a chart of a three month trial treatment of 6 patients illustrating the levels of Lp-PLA2 from the beginning of the treatment trial to the end.

FIG. 5 is a chart from the three month trial of FIG. 4 illustrating the pocket probing depths at the beginning, one month and three month trial dates for the six patients.

FIG. 6 is a chart of the three month trial of FIG. 4 illustrating the bleeding probe depths at the beginning, one month and three month trial dates for the 6 patients.

It should be understood that throughout the drawings, corresponding reference numerals indicate like or corresponding parts and features.

### DETAILED DESCRIPTION

The following description is merely exemplary in nature and is not intended to limit the present disclosure or the disclosure's applications or uses.

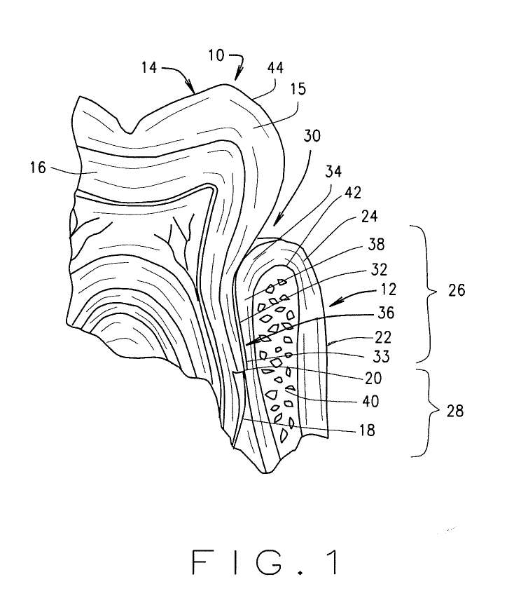

For reference, FIG. 1 shows a tooth 10 with surrounding gum 12 that may have an identified periopathogen. The tooth 10 includes a crown 14 having enamel 15, an inner composition of dentine 16 that forms the root of the tooth 10 , and cementum 18 that covers the outer surface of the lower portions of the tooth 10 below the enamel 15. A cementoenamel junction (CEJ) 20 is defined as the intersection of the lower end of the enamel 15 and the start of the cementum 18. As shown, the gum 12 includes gingiva 22 that has gingival tissue 24 having a portion referred to as marginal gingiva tissue 26, and attached gingiva tissue 28, the two portions generally being separated by the CEJ 20. The gum 12 forms a gingival sulcus 30 at the intersection with the tooth 10. An epithelium 32 covers the lower portions of the enamel 15 above the CEJ 20 such that the cementum 18 is not exposed in a healthy tooth 10 as shown. Connective tissue 34 of the gingiva tissue 28 is adjacent to the epithelium 32 (this portion referred to as the junctional epithelium 33) and includes a bed of capillaries, hereinafter referred to as a capillary bed 36, and alveolar fibers 38. An alveolar bone 40 has an alveolar crest 42 that extends nearly to the CEJ 20 to form a deep socket (not shown) for the tooth 10. Biofilm 44 generally forms to coat the outer surface of the enamel 15.

This disclosure addresses methods and assemblies for treating patient periodontal conditions and is based on an evaluation of such systems and methods on cardiovascular factors and markers such as systemic inflammatory markers including, but not limited to, lipoprotein-associated phospholipase A2 (Lp-PLA2) blood levels and other systemic markers such as, Interleukin 1 (IL-1), Interleukin 6 (IL-6), Interleukin 8 (IL-8), tumor necrosis factor-alpha (TNF-alpha), C-reactive protein, and plasminogen activator inhibitor type-1 (referred herein individually and collectively as “systemic inflammatory markers”, before, during and after such periodontal treatment. This disclosure is based on a pilot study that demonstrated that treating the patient's periodontal conditions resulted in decreasing the periodontal markers and lowering, the IL-1, IL-6, IL-8, TNF-alpha, C-reactive protein Lp-PLA2 levels and others as one exemplary cardiovascular marker and these findings may be important as an adjunct of systemic inflammatory diseases including cardiovascular treatment.

In one embodiment, a method of reducing a systemic inflammatory marker associated with cardiovascular disease in a patient is provided through treatment of periodontal disease that includes performing a medical evaluation examination of the patient that includes, among other processes, the determining of a level of one or more systemic inflammatory markers for cardiovascular disease in the patient.

The method includes comparing the determined level of the systemic inflammatory marker to a predetermined level of the systemic inflammatory marker and where the comparing results in a determined systemic inflammatory marker level being greater than the predetermined level, the process includes performing a periodontal evaluation examination of a patient.

Where or when the comparing results in a determined risk factor level such as level of systemic inflammatory marker is greater than the predetermined level, the method includes performing a periodontal evaluation examination of a patient including evaluating a gum tissue associated with one of more teeth of the patient and identifying an oral periopathogen and a biofilm associated therewith, and identifying one or more treatment regions related thereto. The performing of the periodontal evaluation examination can include taking one or more cultures, each associated with at least one of the treatment regions, and using a suitable identification technique selected from the group consisting of visual analysis using a microscope, chemical analysis, DNA analysis and PCR analysis.

In one example such as when the systemic cardiovascular marker is lipoprotein-associated phospholipase A2 (Lp-PLA2) IL-1, IL-6, IL-8, TNF-alpha, C-reactive protein and others and the determined risk factor level is the level in the blood of the systemic inflammatory marker level, the comparing can be the determined level of said markers.

Such evaluation includes evaluating a gum tissue associated with one of more teeth of the patient and identifying an oral periopathogen and a biofilm and an environment associated with the periopathogen, as well as identifying one or more treatment regions related to the identified oral periopathogen and the biofilm.

The method further includes preparing a periodontal medicament delivery tray for the patient with the delivery tray having at least one application region including at least one tooth indentation for direct application of one or more medicaments to at least one of the identified application regions.

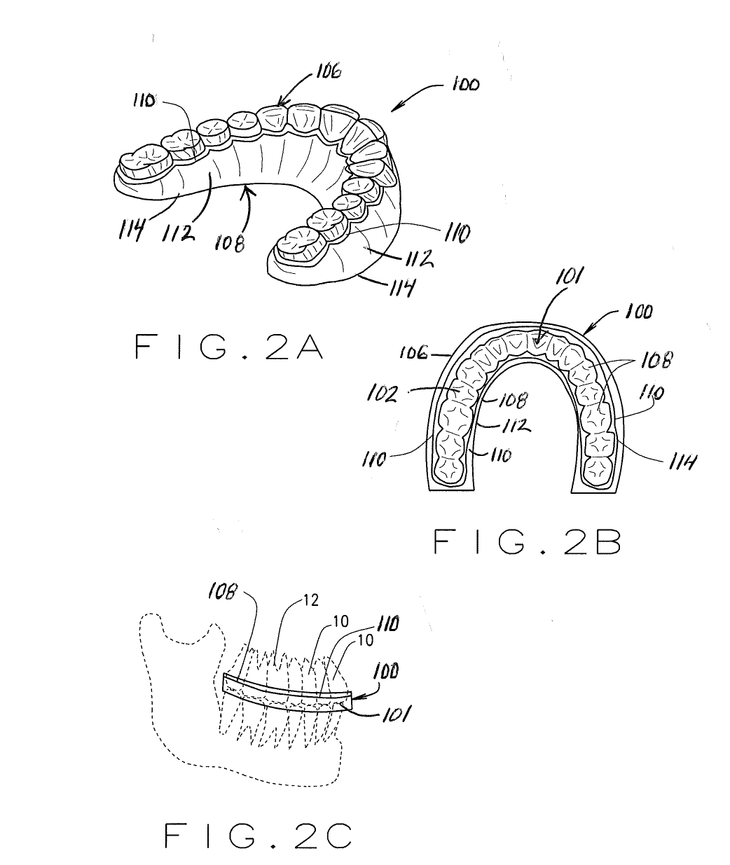

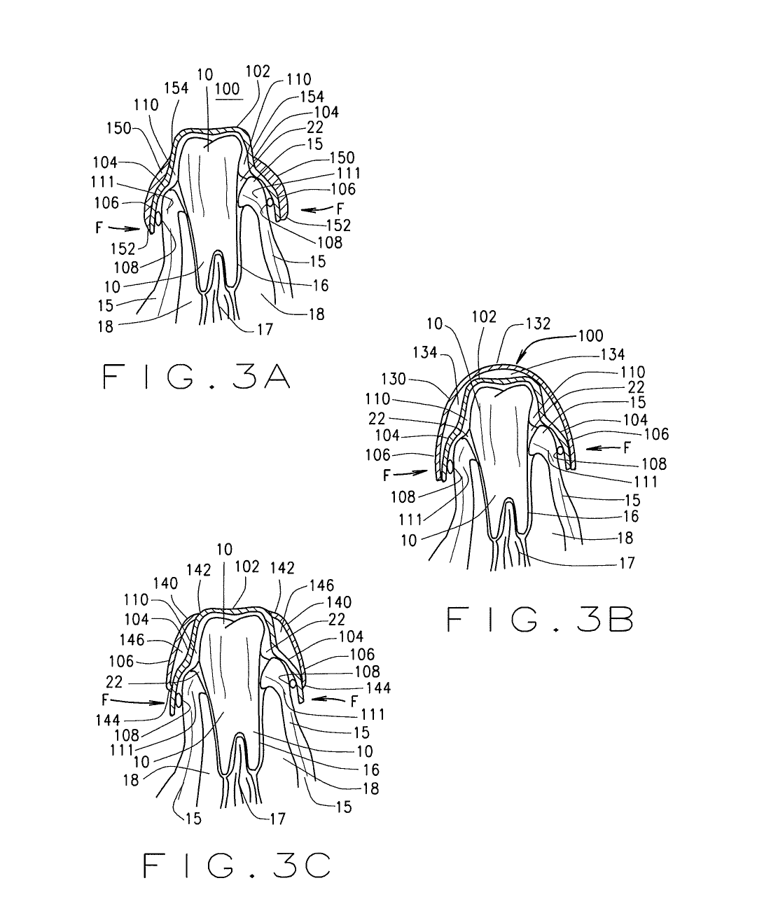

One example of a suitable medicament delivery tray is shown in FIGS. 2A and 2B, one of which that is well suitable is the Perio Tray™ as described in this inventors U.S. Pat. No. 6,966,773. As described herein, such periodontal delivery trays, or variations thereof, are configured to be worn by the patient for the purpose of delivery of the medicament for not only controlling the oral periopathogens but also for reducing the systemic inflammatory markers such as those associated with cardiovascular disease. Referring to FIGS. 2A and 2B, a form-fitted flexible periodontal medicament delivery tray 100 is prepared having a body 102, having walls 104 forming skirts 106. and having chambers 110 that are medicament delivery chambers for receiving the selected medicament 101 and formed to receive one or more teeth 10 when placed on the patient. Tray 100 can be formed to have a body 102 composed of a suitable soft plastic elastomeric or other suitable material which is molded in place to the patient's teeth 10 as shown is FIG. 2A so as to form a dental arch recess 120 that conforms closely to a patient's teeth 10. Tray 100 is shown is FIGS. 2A, 2B and 2C to be a full arch tray 100, but those skilled in the art will recognize that a partial arch tray or a dual arch tray may be used, if desired. Further, the tray can be formed to include a raised seal lip 108 along an inner skirt 106 or wall 104 and an outer skirt 106 or wall 104 such that the raised seal lip 108 is set apart from the teeth 10 but along the gum line 111 (See FIG. 3A) associated with the teeth 10 such that the raised seal lip does not substantially contact the teeth 10.

One exemplary tray 100 can include a static raised seal that can be formed in a location along the gum line of the patient such from a resilient material so that the seal forms against the gum when the tray is placed about the teeth and so that the tray does not contact the teeth themselves. Other dynamic positive and negative pressure trays are also possible. FIG. 2 is a form-fitted flexible periodontal medicament delivery tray 100 having a raised inner seal 100 that is adapted for applying one or more of the medicaments 102 described in the various methods in accordance with various embodiments. In other examples of suitable trays, U.S. Pat. No. 8,591,229 provides various embodiments, some of which are illustrated in FIG. 3 (including FIGS. 3A, 3B, and 3C), which are also shown by way of example. Reference to U.S. Pat. No. 8,591,229 is made and incorporated herein for this embodiment rather than repeating that disclosure.

As noted above, the method includes preparing a periodontal medicament delivery tray 100 for the patient where the prepared delivery tray is provided with at least one application region 110 including at least one tooth indentation for direct application of a medicament to at least one of the application regions 110. Various trays 100 are possible, but as it is very desirable to deliver as much of the selected medicament into the sulcus or as deep into the periodontal pocket as possible, including a deep pocket that is greater than 6 mm in depth, the prepared tray should be capable of creating a pressure within the cavity of the tray 100 that forces the medicament deep into the sulcus or pockets.

In another embodiment, FIG. 3A illustrates a tray 100 having a skirt 106 with an outer layer 150 that is formed along or on the outer surface of a wall 104 of body 102 without forming any type of skirt chamber. The outer layer 150 has a lower end 152 and an upper end 154 with the outer layer 150 being attached to the outer surface of the wall 104 from the upper end 154 to the lower end 152. Of course this attachment can be by way of co-forming or manufacturing. The skirt 106 formed on the outer surface of the wall 104 essentially creates a bilaminar formation, with the wall 104 being formed of one material and the outer layer 150 forming the skirt 106 being that of the described skirt material. In this embodiment, the outer layer 150 or skirt 106 provide the stiffness and biasing to the wall 104 and to the seal 108 towards to the gum line 111.

In yet another embodiment, FIG. 3B illustrates a tray 100 with a skirt 106 is constructed to have a monolayer outer body 130 positioned outside of body 102 that forms a chamber 110 and includes seals 108. In such embodiments, the skirt 106 that provides a biasing force F is a part of or formed by the monolayer outer body 130. In this manner, the body 102 forming the chamber 110 and having the seals 108 can be made of a consistent material, such as one ideally suited for forming to the contours of each patient's teeth and gum line. In some embodiments, the monolayer outer body 130 is formed as an external separate surface structure that can be attached or just placed over the body 102 before or after the body 102 is placed on the teeth 10 and gum tissue 11 to be treated. This could be referred to as a two-piece embodiment. In other embodiments, the monolayer outer body 130 can be attached or otherwise formed or constructed as a unibody with or on the external surfaces of the outer wall 104 of the body 102. In some embodiments, a skirt chamber 134 or ancillary chamber is formed between the outer body 130 and body 102 along one or more portions of the outer surface area of the body 102. The skirt chamber 134 may be filled or be a void.

The monolayer outer body 130 can be formed of an elastic resilient material and dimensioned to the walls 104 of the main body so that the skirt 106 of the monolayer outer body 130 can apply a pressure F to the wall 104 proximate to the seal 108. This can be preferably a consistent amount of pressure F along the length of the seal 108 and gum line 111.

As shown the monolayer outer body 130 defines the skirt 106 having the inner skirt 106 outside of the inner wall 104 and the outer skirt 106 outside of the outer wall 104. The monolayer outer body 130 also includes a top connecting portion 132 connecting the two opposing skirts 106. The top connecting portion 132 can also add to the resilience and aid the skirts 106 in applying the biasing force F. As shown, the skirt 106 engages the outer surface of the outer and inner walls 104 defining the channel 110 therebetween to apply the biasing force F against each to restrict the outer movement of the walls 104 and/or to apply force F to each wall 104 and therefore to the outer and inner seals 108 along the gum line 111. The applied biasing force F of the skirt 106 of the monolayer outer body 130 increases the air tightness of the seal 108 against the gum line 111. While not shown in FIG. 3B, one or more pressure ports can be utilized with this embodiment so as to increase or decrease the pressure within the medicament chamber 110.

In still another embodiment, FIG. 3C illustrates a tray 100 can produce a positive pressure with skirt 106 attached at one or more points on the outer surfaces of the walls 104 of the body 102 including the inside wall 104 and the outside wall 104. This embodiment differs from the embodiment of FIGS. 3A and 3B as the skirt 106 is formed by outer layer 140 of wall 104 and does not include a full separate body 130 and does not include the top connecting portion 132. The outer layer 140 is composed of the resilient stiffened material for the skirt as described above.

The inner and outer skirts 106 of this embodiment are separate and not attached or connected and do not form a single unibody construction that covers the entire outer top surface of the body 102. As such, the outer layer 140 has an upper end 142 and a lower end 144, one or both of which can be attached to the body 102. In this embodiment, a skirt chamber 146 is formed between the outer surface of wall 104 and the inner surface of the skirt 106 which is formed by the outer layer 140. The skirt chamber 146 can be filled as described above or can be a void. The upper end 142 is attached to the wall 104 below a top of the body 102 of tray 100, but the position of such attachment can vary from that illustrated and still be within the scope of the present disclosure.

The orientation and position of the lower end 144 of skirt 106 can be different in slightly different embodiments. FIG. 3C illustrates a tray 100 having the lower end 144 of the skirt 106 or outer layer 140 positioned below or beyond the position of the seal 108 and/or the gum line 111. As shown, the lower end 144 can extend down the wall 104 to a point in close proximity to an end of the wall 104. The lower end 144 can be attached to the wall 104 at this point or the lower end 144 along with the other portions of the outer layer 140 can be formed with the body 102. In this embodiment, the lower end 144 of the skirt 106 exerts the biasing force F to the lower end of wall 104 and therefore biases or applies pressure to the seal 108 to the gum line 111.

Other examples of suitable medicament delivery trays 100 are also possible, and these are only examples.

Next, the method includes selecting a medicament for placement in one of the application regions of the periodontal medicament delivery tray where selecting is a function of a efficacy (known, predetermined or discovered) of the medicament in regard to the identified oral periopathogen and biofilm of the gum tissue associated with the one application region in which the medicament is to be placed. The selected medicament can include one or more agents, and includes at least one oxygenation agent among such one or more agents.

The method also includes placing the selected medicament into the associated application region of the prepared tray 100, applying the tray 100 with the placed selected medicament to the treatment region of the patient for a predetermined period of time such that the medicament is directly delivered or applied against the gum tissue of the treatment region and at least a portion of the selected medicament is forced into a gingival sulcus or periodontal pocket of the gum tissue of the treatment region. This is shown in FIG. 2C.

The method also includes selecting a medicament for placement in at least one of the application regions of the periodontal medicament delivery tray, the selected medicament including at least one oxygenation agent, but could also include an antibiotic and/or an antimicrobial agent. The selection of the oxygenation agent can be based on various properties of the agent and in some embodiments is based on the ability of the agent to decrease systemic inflammatory markers in the blood. Also in some, it can be based on the agent's ability to penetrate the biofilm.

The medicament can also include an antibiotic and/or an antiviral agent or other forms of medicaments that are determined to reduce the inflammatory and cardiovascular marker such as an ability to decrease the LP-PLA-2 levels in the blood. One antibiotic is doxycycline but others are also possible. The oxygenation agent can be a hydrogen peroxide medicament such as a hydrogen peroxide gel and one such gel has about 1.7 percent concentration.

The selection process can also include selecting at least one of an antimicrobial agent and an antibiotic as a function of at least one of an efficacy against the determined oral periopathogen and/or an ability of the antibiotic to penetrate the determined biofilm.

The method further includes placing each selected medicament to the associated application region of the prepared tray and applying the tray with the placed medicaments to the treatment region of the patient for a predetermined period of time. The process of applying includes directly delivering the placed medicaments against the gums of the treatment region and forcing at least a portion of the medicaments into a gingival sulcus or periodontal pocket of the gum tissue of the treatment regions. The applying can include directly delivering at least a portion of the medicaments placed in the tray into periodontal pockets of the gum that is placed for a predetermined period of time and frequency of placing based on the depth of the periodontal pocket containing the identified periopathogen. For instance, and only by way of example, if the depth is greater than about 6 mm, the applying could be repeated 3 to 6 times (and maybe 4) per day and the predetermined time for each applying is about 15 minutes. If and when the periodontal pocket depth is between about 3 mm and about 6 mm, the applying can be repeated 3 times per day, and if and when the depth is below 3 mm, the applying can be only repeated 2 times per day and the predetermined time for each applying is about 15 minutes. The 15 minutes identified herein can be adjusted based on various factors including the selected medicaments.

As with most treatments, a single treatment is not enough and as such, the method includes repeating the performing of a medical evaluation examination, the comparing selecting, placing and applying process steps until at least one occurs, a) the comparing results in the determined systemic inflammatory marker level being less than or equal to the predetermined level, b) during the periodontal evaluation examination the evaluating of the gum tissue identifies the oral periopathogen is less than a predetermined level, c) during the periodontal evaluation, it is determined that the identified environment for the identified periopathogen has been substantially altered from the first performed medical evaluation examination.

The processes of selecting, placing and applying are repeated and often the performing of a medical evaluation examination is repeated therewith until at least one treatment milestone is reached. These include, but are not limited to the comparing results in the determined risk factor level being less than or equal to the predetermined level that is predetermined to be desired to be indicative of being beneficial, that during the periodontal evaluation examination the evaluating of the gum tissue identifies the oral periopathogen is less than a predetermined level, and the during the periodontal evaluation, it is determined that environment for the identified periopathogen has been substantially altered. For example, this could be from an anaerobic environment to an aerobic one, or the reduction in the depth of the periodontal pocket containing the environment, by ways of example. In one embodiment, the environment can be identified where the identified or determined periopathogen has been substantially altered or where the periodontal pocket depth containing the identified periopathogen has been reduced to less than 3 mm, or possible less than 2 mm.

In one procedural or treatment process, the processes of placing and applying are applied as follows: a) during the periodontal evaluation examination, identifying periodontal pockets of a depth greater than 6 mm. In such cases, the step of applying includes directly delivering at least a portion of the medicaments placed in the tray into periodontal pockets of the gum having the depth of greater than 6 mm and the placing and applying can be repeated such as 4 times per day and the predetermined time for each applying is about 15 minutes (dependent on the medicament that is selected). During a subsequent periodontal evaluation examination, if it is identified the periodontal pockets have a reduced depth between about 3 mm and less than or equal to 6 mm, the applying can be reduced to about 3 times per day and if and when reduced depth is less than about 3 mm, the applying can be only repeated 2 times per day.

As should be understood, this method can be supplemented, before or during one or more repetitions of the one or more of the steps, with a one or more scaling and root planning (SRP) treatments or surgery as may be determined by a dentist. For instance, an SRP can be performed as an initial step before the initial placing and applying or can be performed after an initial series of such placings and applyings, which are then continued after such SRP.

In another embodiment, a method of lowering systemic inflammation, including lipoprotein-associated phospholipase A2 (Lp-PLA2) level in blood of a patient includes performing a medical evaluation examination of a patient that includes determining a level in the blood of the patient of systemic inflammatory markers including IL-1, IL-6, IL-8, TNF-alpha, C-reactive protein, lipoprotein-associated phospholipase A2 (Lp-PLA2) level and comparing the determined level of IL-1, IL-6, IL-8, TNF-alpha, C-reactive protein, Lp-PLA, and others to a predetermined level thereof. In such embodiments, the comparing of results can be a comparing of the determined level of IL-1, IL-6, IL-8, TNF-alpha, C-reactive protein. Lp-PLA2 and others to a predetermined desired or objective or threshold level (generally described herein as the predetermined level. The comparing process can be a determining that the determined level is greater than the predetermined level. When such determination is made, then the performing of a periodontal evaluation examination of a patient including evaluating a gum tissue associated with one of more teeth of the patient and identifying an oral periopathogen and a biofilm associated therewith, and identifying one or more treatment regions related thereto. Of course if this process step had already been prepared, this step can be skipped initially or in one or more repetition during such treatment.

In one embodiment, to begin the treatment, the periodontal medicament delivery tray is prepared for the patient for the determined or identified treatment regions. The delivery tray has one or more application region with each including at least one tooth indentation for direct application of a medicament to at least one of the application region that is associated with one or more treatment regions. Of course if a medicament delivery tray is already available for the patient, this step can be omitted or skipped in the initial or subsequent repetitions.

This particular process can include, as also addressed above, selecting a medicament for placement in at least one of the application regions of the periodontal medicament delivery tray. The selected medicament can include one or more oxygenation agents, an antibiotic and an antiviral agent. Such selected medicament is placed into the application regions for which the medicament is selected for the particular treatment region. The tray with the placed medicaments is applied to the application and associated treatment regions so that the placed medicaments are against the gums of the treatment region and forced at least in portion into the gingival sulcus area or periodontal pocket of the gum tissue of the treatment regions. This placing and applying is repeated numerous times such as numerous times a day and for a predetermined period of time for each applying.

These processes are repeated in various combinations including repeating the performing of a medical evaluation examination and the comparing selecting, placing and applying process steps (as well as the preparing process if a new tray is required) until it is determined that a success, and full treatment has occurred. For example, this can be when the comparing results in the determined level of Lp-PLA2 being less than or equal to the predetermined level. In another instance this can be during the periodontal evaluation examination if and when the evaluating of the gum tissue identifies the oral periopathogen is less than a predetermined level or has been eliminated. In other instances this can be during the periodontal evaluation, it is determined that environment for the identified periopathogen has been substantially altered, such as to an aerobic one.

In some embodiments, the systemic inflammatory and cardiovascular marker is IL-1, IL-6, IL-8, TNF-alpha, C-reactive protein , lipoprotein-associated phospholipase A2 (Lp-PLA2) and others and the determined risk factor level is the level in the blood of the patient of IL-1, IL-6, IL-8, TNF-alpha, C-reactive protein, lipoprotein-associated phospholipase A2 (Lp-PLA2) and other systemic inflammatory marker levels. The comparing can be a comparing of the determined level of IL-1, IL-6, IL-8, TNF-alpha, C-reactive protein, Lp-PLA2 ;and others to a predetermined level thereof. This selecting can include selecting the oxygenation agent includes selecting as a function of ability to decrease the IL-1, IL-6, IL-8, TNF-alpha, C-reactive protein LP-PLA-2 and other levels in the blood.

In some embodiments, the oxygenation agent can be any such agent but in one embodiment is hydrogen peroxide. The selected antibiotic can be any antibiotic and in one embodiment is a broad spectrum antibiotic such as doxycycline. In some embodiments, the selecting also includes selecting the antibiotic as a function of ability to decrease the IL-1, IL-6, IL-8, TNF-alpha, C-reactive protein, LP-PLA-2 levels in the blood. The selecting can also include selecting the antibiotic as a function of an efficacy of the antibiotic medicament against the determined oral periopathogen. In other embodiments, the selecting can include selecting at least one of an antimicrobial agent and an antibiotic, and the selecting can be a function of the ability to penetrate the determined biofilm.

Medicament delivery according to this disclosure provides for direct application and/or delivery to the gingival sulcus area or periodontal pocket of the gums where one or more periopathogens have been identified or determined which can cause increased levels of systemic inflammatory markers including cardiovascular markers such as Lp-PLA2 levels IL-1, IL-6, IL-8, TNF-alpha, C-reactive protein, or other determinable risks of cardiovascular disease.

Increased blood levels of IL-1, IL-6, IL-8, TNF-alpha, C-reactive protein ,Lp-PLA2 have been related to an increased risk of cerebral thrombosis initial and recurrent coronary events, poor outcomes after acute coronary events and cardiovascular disease associated with metabolic syndrome. There is some controversy regarding the possible affect IL-1, IL-6, IL-8, TNF-alpha, C-reactive protein, Lp-PLA2 may have on reducing atherogenesis by decreasing oxidized low-density lipoproteins (oxLDL) and platelet-activating factor (PAF), as degradation of these molecules may also have proinflammatory, proliferative and proatherogenic properties. Because IL-1, IL-6, IL-8, TNF-alpha, C-reactive protein ,Lp-PLA2 levels relate to an increased risk to the patient, it would appear that reducing IL-1, IL-6, IL-8, TNF-alpha, C-reactive protein ,Lp-PLA2levels would be a beneficial goal of reducing systemic inflammation including cardiovascular therapy.

Systemic inflammatory markers such as IL-1, IL-6, IL-8, TNF-alpha, C-reactive protein, Lp-PLA2 levels and others have been found to be elevated in chronic periodontal patients when compared to healthy control subjects. Research from this study positively associated serum c-reactive protein and Lp- PLA2 levels and bleeding upon probing and periodontal inflammation prompting the question whether periodontal disease treatment could reduce the risk of cardiovascular disease in chronic periodontal patients.

A pilot experimental use study of the present method and assemblies was undertaken with a control group of a limited number of patients to determine the extent that treating a patient's identified periopathogen with the described Perio Protect Method will beneficially affect the patient's systemic IL-1, IL-6, IL-8, TNF-alpha, C-reactive protein ,Lp-PLA2 levels. The described Perio Protect Method as applied in this study and described herein utilized the benefits of a prescription medicament delivery tray (Perio Tray®, Perio Protect LLC, St. Louis, MO www.perioprotect.com) to deliver medications into shallow (<5 mm) and deep (>5 mm) pockets in combination with mechanical debridement, scaling and surgery as required. As one example, in a six (6) month study of patients with moderate to severe periodontal disease, a 1.7 percent hydrogen peroxide gel (Perio Gel®) in a prescription customized tray (Perio Tray®) was used as an adjunct to scaling and root planning (SRP). This experimental study demonstrated that in just over six months there was a clinically significant improvement in the reduction of pocket depths and bleeding indices when compared to treatment by SRP alone. The result also indicated that the effect did not appear to be transitory. The test group reductions from baseline were maintained for six months. This pilot study evaluated whether the method not only treated the periodontal disease but also the was capable of reducing systemic inflammation factors such as c-reactive protein and LpPLA2 levels as each patient selected for this study also had some form of cardiovascular disease.

This pilot experimental use study was an initial attempt to demonstrate the benefit of treating periodontal disease with the Perio Protect Method and its ability to reduce a patient's IL-1, IL-6, IL-8, TNF-alpha, C-reactive protein, Lp-PLA2 systemic inflammatory markers. The Perio Protect Method® can in some cases be combined with SRP and/or surgery with the use of a custom formed medical device (Perio Tray®) to deliver the doctor selected medications into the gingival sulcus or periodontal pocket. The tray delivery can, in some cases, be used by the patients at home under the dentist prescription in accordance with the periodontal conditions and treatments are modified as healing occurred over time.

The medications used in the pilot study were 1.7% hydrogen peroxide gel (Perio Gel®) and 1-3 drops/tray Vibramycin Syrup [50 mg/5m1 doxycycline]. The medications replaced by the patient into the Perio Tray and delivered into the periodontal pocket in accordance with the scope and magnitude of the patient's periodontal disease. The Perio Protect Protocol was determined by the patient deepest periodontal pockets as follows:

| | |
|---|---|
| >6 mm pockets | 4 times/day at 15 minutes |
| 3 mm-6 mm pockets | 3 times/day at 15 minute |
| <3 mm pockets (maintenance) | 2 times/day at 15 minutes |

The Silver Staff Method of evaluation was used to measure the patient's Lp-PLA2 levels. The Silver Staff Method involved having the patient warm the patient's hand in warm tap water for 10 minutes. A finger was then cleansed with alcohol and a finger stick was performed at the edge of the fingerprint whorl. The patient's name and identification number were placed on the collection card. Drops of blood were then placed on the collection card until sufficient blood was accumulated to pass a registration line. The card was then processed as per the manufacturer's instructions and the card/sample was then mailed to Silver Staff for evaluation. The Silver Staff evaluators were blinded to the samples and testing as they only evaluated the blood sample results.

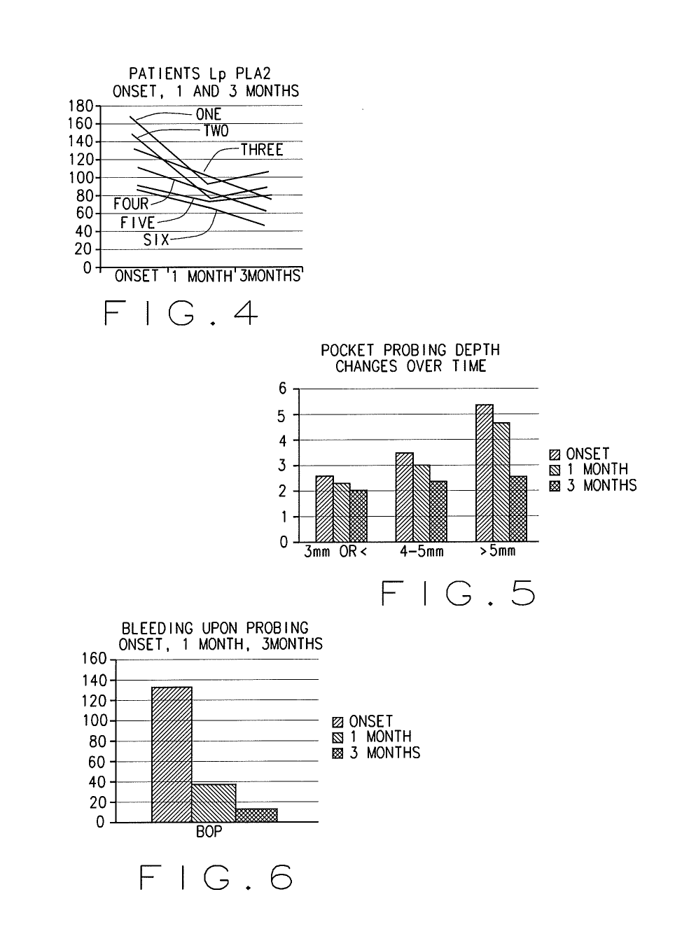

Six patients without experiencing a cardiovascular event were accepted into this case study. Each of the patients had active periodontal disease and agreed to have their Lp-PLA2 levels evaluated at onset, at one and three months. The acceptance criteria were the patient could not be under active cardiovascular treatment and could not take any cardiovascular or oral antibiotic medications during the course of the three month pilot study as shown in FIG. 4.

The patients started their three month periodontal disease treatment with Perio Tray administration of <1 gm 1.7% hydrogen peroxide and one to three drops Vibramycin Syrup/ tray. Following two weeks of Perio Tray usage scaling and root planning and/or debridement were completed. One of the six patients also received site specific surgery as needed. The patients were re-examined at one month for changes in their periodontal conditions and the blood samples were again taken and measured with the Silver Staff Method to evaluate their Lp-PLA2 levels. Alterations in the frequency of Perio Tray delivery were made in accordance with the treatment progress and/or new trays were fabricated as required by the healing that occurred.

The initial results were maintained by using the Perio Tray deliver of both medicaments one to two times a day for the remaining 3 month study period. At the three month visit, the periodontal conditions and blood samples were taken to evaluate the patient's Lp-PLA2 levels. The patients completed the three month case study without any complications or adverse situations.

In the described experimental test, a total of 6 subjects completed the 3 month trial of FIG. 4. After 4 weeks of periodontal treatment, analysis of deeper pockets (>5 mm at baseline) showed that pocket probing depth (PPD) decreased from baseline by more than 0.90 mm. As shown in FIG. 5, at three months mean PPD decreases were 1.70 mm for the pilot study group. Analysis of shallower pockets (i.e., <5 mm at baseline) showed 0.57 mm reduction at one month and 1.08 mm reduction at three months.

As shown in FIG. 6, bleeding upon probing (BOP) was recorded as the number of bleeding sites recorded after probing the mesial buccal, buccal and distal buccal, mesial lingual, lingual and distal lingual aspects of each tooth. The reduction in bleeding for the test group showed a clinical improvement for both deep and shallow pockets (FIG. 6). The mean BOP reductions for PPDs >5 mm was approximately 62% at one month and an 80% decrease at three months. For PPDs in the <5 mm range, there was a 74% decrease in bleeding upon probing at one month and almost a 90% decrease in the number of bleeding points at three months (FIG. 6). The data indicate that prescription tray delivery of 1.7% hydrogen peroxide gel and Vibramycin resulted in a greater reduction of bleeding points in shallow pockets.

Patient's Lp-PLA2 levels were measured as ng/mL prior to treatment and at one and three months of periodontal treatment. The mean Lp-PLA2 levels before treatment were 120.5 ng/ml. The one month Lp-PLA2 levels were 80.7 ng/ml and the three month levels were 76.8mg/m. This equates to a 30.7% decrease in Lp-PLA2 levels at one month and a 37.1% decrease in Lp-PLA2 levels at three months. Individual patient results are indicated in FIG. 4.

The effectiveness of periodontal disease treatment using the Perio Protect Method's combination of mechanical therapy with prescription tray delivery of medications was evaluated in subjects with chronic periodontitis to determine if treating their periodontal conditions would affect their systemic Lp-PLA2 inflammatory markers. The results demonstrated the periodontal treatment was effective in reducing pocket depths and bleeding upon probing (FIGS. 5 and 6) corroborating the findings of earlier trials. The results also show reductions in baseline Lp-PLA2 scores for each patient.

It was hypothesized that treating periodontal disease would decrease patients IL-1, IL-6, IL-8, TNF-alpha, C-reactive protein, Lp-PLA2 levels. Patient's Lp-PLA2 levels were evaluated at the onset of the study and at one and three months of using the Perio Protect Method with the custom formed Perio Tray. The mean Lp-PLA2 level prior to treatment was found to be a mean of 120.5 ng/ml. The Lp-PLA2 levels decreased to a mean of 76.8 ng at one month and further decreased to a mean of 80.7 ng at three months. This equates to a 30.7% reduction in Lp-PLA2 levels in one month and a 37.1% reduction by the third month (FIG. 4). The only treatments rendered during the course of this study were dental procedures using the Perio Protect Method to address the patient's periodontal conditions. All patients were compliant in their periodontal treatments. Improvements in the patient's periodontal conditions (pocket probing depth and bleeding upon probing) appeared to coincide with patient's improvements in Lp-PLA2 levels, but the pilot study involved only 6 patients, which is not a significant number of patients for statistically significant findings to be determined.

This disclosure is based on the results of an experimental use pilot study that resulted in unique findings where six experimental use patients with periodontal disease were treated with the described method under the direct supervision and control of a doctor and their systemic inflammatory markers levels improved as their periodontal conditions improved with the periodontal treatments. The results of this study appears to provide a novel new method of improving a patient's systemic inflammatory markers such as, by way of example, Lp-PLA2 levels, through treating periodontal disease may correlate with improved patients systemic inflammatory markers levels, that can reduce the risk of cardiovascular disease in the patient.

When describing elements or features and/or embodiments thereof, the articles “a”, “an”, “the”, and “said” are intended to mean that there are one or more of the elements or features. The terms “comprising”, “including”, and “having” are intended to be inclusive and mean that there may be additional elements or features beyond those specifically described.

Those skilled in the art will recognize that various changes can be made to the exemplary embodiments and implementations described above without departing from the scope of the disclosure. Accordingly, all matter contained in the above description or shown in the accompanying drawings should be interpreted as illustrative and not in a limiting sense.

It is further to be understood that the processes or steps described herein are not to be construed as necessarily requiring their performance in the particular order discussed or illustrated. It is also to be understood that additional or alternative processes or steps may be employed.

What is claimed is:

1. A method of reducing a systemic inflammatory marker associated with cardiovascular disease in a patient through treatment of periodontal disease, the method comprising:
performing a medical evaluation examination of the patient that includes determining a level of the systemic inflammatory marker for cardiovascular disease in the patient;
comparing the determined level of the systemic inflammatory marker to a predetermined level of the systemic inflammatory marker;
where the comparing results in a determined systemic inflammatory marker level being greater than the predetermined level, performing a periodontal evaluation examination of a patient including evaluating a gum tissue associated with one of more teeth of the patient and identifying an oral periopathogen and a biofilm and an environment associated with the periopathogen, and identifying one or more treatment regions related to the identified oral periopathogen and the biofilm;
preparing a periodontal medicament delivery tray for the patient, the delivery tray having at least one application region including at least one tooth indentation for direct application of one or more medicaments to at least one of the identified application regions;
selecting a medicament for placement in one of the application regions of the periodontal medicament delivery tray, the selecting being a function of a predetermined efficacy of the medicament in regard to the identified oral periopathogen and biofilm of the gum tissue associated with the one application region in which the medicament is to be placed, the selected medicament including at least one oxygenation agent;
placing the selected medicament into the associated application region of the prepared tray;
applying the tray with the placed selected medicament to the treatment region of the patient for a predetermined period of time, the applying including directly delivering the placed selected medicament against the gum tissue of the treatment region and forcing at least a portion of the selected medicament into a gingival sulcus or periodontal pocket of the gum tissue of the treatment region;
repeating the performing of a medical evaluation examination, the comparing, the selecting, the placing and the applying process steps until at least one occurs, a) the comparing results in the determined systemic inflammatory marker level being less than or equal to the predetermined level, b) during the periodontal evaluation examination the evaluating of the gum tissue identifies the oral periopathogen is less than a predetermined level, c) during the periodontal evaluation, it is determined that the identified environment for the identified periopathogen has been substantially altered from the first performed medical evaluation examination.

2. The method of claim 1 wherein selecting the medicament includes selecting at least the medicament and one or more agents of the medicament based on an ability of the antibiotic to penetrate the determined biofilm.

3. The method of claim 1 wherein selecting the medicament includes selecting at least one of an antimicrobial agent and an antibiotic agent and wherein such selecting the medicament is a function of an ability of the antibiotic to penetrate the determined biofilm.

4. The method of claim 1 wherein selecting the medicament includes selecting the oxygenation agent and selecting an antibiotic wherein the selecting of the oxygenation agent is a function of the oxygenation agent's ability to penetrate the determined biofilm.

5. The method of claim 4 wherein the antibiotic is doxycycline.

6. The method of claim 1 wherein the determined level of the systemic cardiovascular marker is lipoprotein-associated phospholipase A2 (Lp-PLA2) and the determined systemic inflammatory marker level is the level in the blood of the patient of lipoprotein-associated phospholipase A2 (Lp-PLA2) level and wherein comparing is comparing of the determined level of Lp-PLA2 to a predetermined predetermined level thereof.

7. The method of claim 6 wherein selecting the oxygenation agent includes selecting as a function of ability to decrease the Lp-PLA-2 levels in the blood.

8. The method of claim 6 wherein selecting the medicament includes selecting an antibiotic, wherein such selecting is a function of ability to decrease the Lp-PLA-2 levels in the blood.

9. The method of claim 8 wherein the antibiotic is doxycycline.

10. The method of claim 1 wherein determining a level of a systemic inflammatory marker for cardiovascular disease includes selecting the systemic inflammatory marker from the group consisting of lipoprotein-associated phospholipase A2 (Lp-PLA2), Interleukin 1 (IL-1), Interleukin 6 (IL-6), Interleukin 8 (IL-8), tumor necrosis factor-alpha (TNF-alpha), C-reactive protein, and plasminogen activator inhibitor type-1.

11. The method of claim 1 wherein performing the periodontal evaluation examination includes taking one or more cultures, each associated with at least one of the treatment regions and using a suitable identification technique selected from the group consisting of visual analysis using a microscope, chemical analysis, DNA analysis and PCR analysis.

12. The method of claim 1 wherein selecting the medicament that includes at least one oxygenation agent is selecting a hydrogen peroxide medicament.

13. The method of claim 12 wherein the hydrogen peroxide medicament is a hydrogen peroxide gel of about 1.7 percent.

14. The method of claim 1 wherein the applying includes directly delivering at least a portion of the medicaments placed in the tray into periodontal pockets of the gum having a depth of greater than about 6 mm and wherein the placing and applying are repeated 4 times per day and the predetermined time for each applying is about 15 minutes.

15. The method of claim 1 wherein the applying includes directly delivering at least a portion of the medicaments placed in the tray into periodontal pockets of the gum having a depth of between about 3 and equal to or less than about 6 mm and wherein the placing and applying are repeated 3 times per day and the predetermined time for each applying is about 15 minutes.

16. The method of claim 1 wherein the applying includes directly delivering at least a portion of the medicaments placed in the tray into periodontal pockets of the gum having a depth of less than about 3 and wherein the placing and applying are repeated 2 times per day and the predetermined time for each applying is about 15 minutes.

17. The method of claim 1 wherein the processes of placing and applying are applied as follows:
during the periodontal evaluation examination, identifying periodontal pockets of a depth greater than about 6 mm, wherein the applying includes directly delivering at least a portion of the medicaments placed in the tray into periodontal pockets of the gum having the depth of greater than about 6 mm and wherein the placing and applying are repeated 4 times per day and the predetermined time for each applying is about 15 minutes;
then during a subsequent periodontal evaluation examination, identifying the periodontal pockets have a reduced depth between about 3 mm and less than or equal to about 6 mm, wherein the applying includes directly delivering at least a portion of the medicaments placed in the tray into periodontal pockets of the gum having the depth between about 3 mm and less than or equal to about 6 mm and wherein the placing and applying are repeated 4 times per day and the predetermined time for each applying is about 15 minutes;
then during another subsequent periodontal evaluation examination, identifying the periodontal pockets have a reduced depth less than about 3 mm, wherein the applying includes directly delivering at least a portion of the medicaments placed in the tray into periodontal pockets of the gum having the depth of less than about 3 mm and wherein the placing and applying are repeated 2 times per day and the predetermined time for each applying is about 15 minutes.

18. The method of claim 1, further comprising also performing one or more scaling and root planning (SRP) of the treatment region before or during the repeating step.

19. The method of claim 1 wherein preparing the delivery tray further includes forming a raised seal in a location such that a resilient material is formed as a seal against the patient's gums, said seal formed so that the seal has substantially no contact with the patient's teeth.

20. The method of claim 1 wherein the environment of identified periopathogen has been substantially altered when a periodontal pocket containing the identified periopathogen has been reduced to less than about 3 mm.

21. A method of lowering lipoprotein-associated phospholipase A2 (Lp-PLA2) level in blood of a patient, comprising:
performing a medical evaluation examination of a patient that includes determining a level in the blood of the patient of lipoprotein-associated phospholipase A2 (Lp-PLA2) level;
comparing the determined level of Lp-PLA2 to a predetermined level of Lp-PLA2;
where the comparing results in the determined level of Lp-PLA2 is greater than the predetermined level of Lp-PLA2, performing a periodontal evaluation examination of a patient including evaluating a gum tissue associated with one of more teeth of the patient and identifying an oral periopathogen and a biofilm and an environment associated with the identified oral periopathogen, and identifying one or more treatment regions related thereto;
preparing a periodontal medicament delivery tray for the patient, the delivery tray having at least one application region including at least one tooth indentation for direct application of a medicament to at least one of the identified application regions;
selecting a medicament for placement in at least one of the application regions of the periodontal medicament delivery tray, the selected medicament including at least an oxygenation agent and an antibiotic, the selecting being a function of a predetermined efficacy of the medicament in regard to the identified oral periopathogen and biofilm;
placing each selected medicament to the associated application region of the prepared tray;
applying the tray with the placed selected medicament to the treatment region of the patient for a predetermined period of time, the applying including directly delivering the placed selected medicament against the gums of the treatment region and forcing at least a portion of the selected medicament into the gingival sulcus area or periodontal pocket of the gum tissue of the treatment regions;
repeating the performing of a medical evaluation examination, the comparing, the selecting, the placing and the applying process steps until at least one occurs, a) the comparing results in the determined level of Lp-PLA2 being less than or equal to the predetermined level; b) during the periodontal evaluation examination the evaluating of the gum tissue identifies the oral periopathogen is less than a predetermined level, c) during the periodontal evaluation, it is determined that identified environment for the identified periopathogen has been substantially altered.

22. The method of claim 21 wherein selecting the medicament includes selecting at least the medicament and one or more agents of the medicament based on an ability of the medicament to penetrate the determined biofilm.

23. The method of claim 21 wherein selecting the medicament includes selecting the oxygenation agent is a function of the oxygenation agent's ability to penetrate the determined biofilm.

24. The method of claim 21 wherein the systemic cardiovascular marker is lipoprotein-associated phospholipase A2 (Lp-PLA2) and the determined systemic inflammatory marker level is the level in the blood of the patient of lipoprotein-associated phospholipase A2 (Lp-PLA2) level and wherein comparing is comparing of the determined level of Lp-PLA2 to a predetermined level thereof.

25. The method of claim 21 wherein selecting the oxygenation agent includes selecting as a function of ability to decrease the Lp-PLA-2 levels in the blood.

26. The method of claim 21 wherein performing the periodontal evaluation examination includes taking one or more cultures, each associated with at least one of the treatment regions and using a suitable identification technique selected from the group consisting of visual analysis using a microscope, chemical analysis, DNA analysis and PCR analysis.

27. The method of claim 21 wherein selecting at least one oxygenation agent is selecting a hydrogen peroxide medicament.

28. The method of claim 21 wherein the selected antibiotic is doxycycline.

29. The method of claim 21 wherein selecting also includes selecting the antibiotic as a function of ability to decrease the Lp-PLA-2 levels in the blood.

30. The method of claim 21 wherein the environment of identified periopathogen has been substantially altered when a periodontal pocket containing the identified periopathogen has been reduced to less than about 3 mm.

31. The method of claim 21, further comprising also performing one or more scaling and root planning (SRP) of the treatment region before or during the repeating step.

32. The method of claim 21 wherein preparing the delivery tray further includes forming a raised seal in a location such that a resilient material is formed as a seal against the patient's gums, said seal formed so that the seal has substantially no contact with the patient's teeth.
